(19) United States
(12) Patent Application Publication
(10) Pub. No.: US 2008/0085548 A1
(43) Pub. Date: Apr. 10, 2008

(54) Transporting Arrangement For Transporting Biomass In A Fermenter For Generating Biogas, And Large-Scale Fermenter For Generating Biogas From Biomass

(75) Inventor: Peter Lutz, Unterföhring (DE)

Correspondence Address: MORRISON & FOERSTER, LLP, 555 WEST FIFTH STREET, SUITE 3500, LOS ANGELES, CA, 90013-1024, US

(73) Assignee: Bekon Energy Technologies GmbH & Co. KG, Landshut (DE)

(21) Appl. No.: 10/591,501

(22) PCT Filed: Mar. 4, 2005
(86) PCT No.: PCT/EP05/02326

§ 371 Date: Aug. 20, 2007

(30) Foreign Application Priority Data
Mar. 4, 2004 (DE) 20 2004 003 398.4

(51) Int. Cl. C12M 1/00; C12M 1/36

(52) U.S. Cl. 435/286.5; 435/290.4

(57) ABSTRACT

The invention provides a transporting arrangement for transporting biomass (22), in particular biomass from energy crops, in a fermenter (2) for generating biogas, it being possible for large-scale fermenters (2) to be operated continuously, in accordance with the principle of the methanation of solid matter, by means of this transporting arrangement. The transporting arrangement (20) comprises a plurality of transporting cushions (24-i) which are arranged one beside the other and have liquid pumped into them, and discharged again, one after the other. This generates a wave movement which transports the biomass (22) through the fermenter (2) from a loading region (10) to an unloading region (14). The invention also specifies a large-scale fermenter having such a transporting arrangement (20).

The invention relates to a transporting arrangement for transporting biomass in a fermenter for generating biogas as claimed in claim 1, and to a large-scale fermenter for generating biogas from biomass as claimed in claim 9.

Up until now, biogas technology has concentrated predominantly on the “wet fermentation” of liquid manure and/or municipal biowastes, in the case of which the biomass is present in liquid, pumpable form. Plants and installations for generating biogas from biomass by the wet-fermentation process are known, for example, from AT 408230 B, WO 96/12789, DE 3228391 A1, AT 361885 B and DE 19746636 A1.

DE 3228391 A1 here discloses a digestion tank in the form of an elastic tube in the case of which the biomass is conveyed through the tube by means of artificially generated peristalsis. The peristaltic movement is generated by means of loops which are drawn over the tube and pulled tight, by means of sleeves which are drawn over the tube and subjected to the action of compressed air, or by means of pistons or plungers which are distributed over the length of the tube and push into the tube one after the other.

DE 19746636 A1 discloses a digestion tank in the form of a round silo, the actual digestion tank being arranged in the center of the round silo and being enclosed by an annular intermediate region.

AT 361885 B likewise discloses a digestion tank in the form of a round silo comprising a cylindrical outer wall and a cylindrical inner wall, as a result of which an annular transporting channel is formed. The liquefied biomass is fed, flows through the transporting channel and is removed again. It is additionally known from AT 361885 B to provide a cylindrical central wall between the inner and the outer walls, an inner transporting-channel ring and an outer transporting-channel ring being formed as result.

Biomass from energy corps with high dry-substance contents (e.g. corn silage) or solid dung can only be added into these “liquid” methods to a limited extent.

So-called “dry fermentation” makes it possible to methanate pourable biomasses from agriculture, biowastes and cultivated municipal areas without converting the materials into a pumpable, liquid substrate. It is possible to ferment biomasses with a dry-substance fraction of up to 50%. This dry-fermentation method is described, for example, in EP 0 934 998.

In the case of “dry” fermentation, the material which is to be fermented is not mixed into a liquid phase, as is the case, for example, with the liquid fermentation of biowastes. Instead, the fermentation substrate which is introduced into the fermenter is kept constantly moist by the percolate being withdrawn from the fermenter base and sprayed over the biomass again. This achieves optimum living conditions for the bacteria. In the case of recirculating the percolate, in addition, the temperature can be regulated, and it is possible to provide additives for process optimization.

Wo 02/06439 discloses a bioreactor or a fermenter in the form of a prefabricated garage which is operated in accordance with the dry-fermentation principle by the so-called batchwise process. Following inoculation with material which has already been fermented, the fermentation substrate is introduced into the fermenter by wheeled loaders. The fermentation tank of garage-like construction is closed by a gas-tight door. The biomass is fermented with the exclusion of air, in which case no further mixing takes place and no additional material is added. The percolate seeping out of the fermentation material is withdrawn via a drainage channel, stored on an intermediate basis in a tank and sprayed over the fermentation substrate again for moistening purposes. The fermentation process takes place in the mesophilic temperature range at 34-37° C., and temperature control takes place by means of floor and wall heating.

DE 3341691 A1 also discloses a process and an installation for the anaerobic treatment of organic substances with a high solids fraction in the case of which biomass is also produced. In this case, the biomass is subjected to a squeezing, peristaltic movement in a channel and thus conveyed through the digestion tank. The peristaltic movement is achieved in that the edges of the channel execute an upwardly and downwardly directed movement parallel to one another. At the same time as the up and down movement, pockets or pouches for generating the squeezing movement are pushed into the channel from beneath.

The biogas produced can be used in a cogeneration plant for the purpose of attaining electric current and heat. In order that there is always enough biogas available for the cogeneration plant, a plurality of fermentation tanks are operated at different points in time in the dry-fermentation plant. At the end of the residence period, the fermenter chamber is completely emptied and then refilled. The fermented substrate is fed for subsequent composting, this resulting in conventional composting of comparable organic manures. Such a plant is being trialed in Munich at present.

A significant disadvantage of the known large-scale fermenters is that they run merely in batchwise operation, i.e. the biogas production of the fermenter has to be interrupted for loading and unloading purposes and the biogas-filled fermenter has to be flooded with air. It would thus be desirable to have a large-scale fermenter in which fresh biomass is fed, and spent biomass is discharged, on a continuous basis without the generation of biogas being interrupted. For this purpose, it is necessary for the large-scale fermenter to be provided with a transporting installation which transports the biomass from a loading region to an unloading region.

WO 93/17091 discloses a closed composting installation in which bubbles which can be subjected to the action of compressed air are arranged in the base of the tank in order for the biomass to be mixed in the tank and in order for it to be transported through the tank from a loading region to an unloading region. On account of the associated leakage problem, this transporting process is not suitable for the generation of biogas with the exclusion of air.

The object of the present invention is thus to specify a suitable transporting installation for transporting biomass in a fermenter for generating biogas, and also a large-scale fermenter having such a transporting installation.

This object is achieved by the features of claims 1 and 9, respectively.

The transporting installation according to the invention comprises a plurality of transporting cushions which are arranged one behind the other in the transporting direction and are successively filled with a liquid and emptied again, thus generating a wave movement which transports the biomass.

Using liquid instead of compressed air reliably prevents harmful air from passing into the digestion tank. Furthermore, using liquid instead of compressed air or gas has the advantage that, on account of the incompressibility of liquids, the volume of the liquid pumped into the respective transporting cushion is equal to the change in volume of the transporting cushion. This results in a defined “transporting displacement” irrespective of the weight of the biomass located above.

It is preferable—claim 2—for the transporting cushions to have their volume increased and reduced again by a percolate. Since a percolate-circulating unit has to be present in any case, the necessary liquid for operating the transporting cushions is preferably taken from this stream of percolate.

According to the further advantageous configuration of the invention from claim 3, the individual transporting cushions are arranged directly in abutment against one another. This assists a quasi-continuous wave movement.

Providing a transporting-cushion covering according to claim 4 prevents biomass from accumulating between the transporting cushions and remaining there. It is possible for the transporting-cushion covering to have an articulated structure or else to comprise just a covering sheet.

The transporting cushions according to the preferred embodiment from claim 5 are arranged and fastened on the base plate of the digestion tank. The up and down movement of the transporting cushions generates the biomass-transporting wave movement over the entire width of the transporting channel.

As an alternative, or in addition, it is also possible for the transporting cushions to be assigned to one another in pairs and arranged opposite one another on the side walls. This also achieves a peristaltic movement of the biomass through the transporting channels—claim 6.

Depending on the dry-substance content of the biomass, it may be advantageous to have a wave movement in the transporting direction or a wave movement in the opposite direction. By virtue of the wave-movement direction being switched over periodically, it is also possible to mix the biomass thoroughly—claims 7 and 8.

This transporting arrangement can be installed in conventional bioreactors or fermenters as are known, for example, from WO 02/06439 A, this resulting in a large-scale fermenter according to the invention as claimed in claim 9. In the case of the large-scale fermenters according to claim 9, fresh biomass is introduced into the large-scale fermenter, which produces biogas on a constant basis, in a loading region via a transfer lock. In the large-fermenter, the biomass is transported from the loading region to an unloading region by the transporting arrangement. As the biomass is transported, biogas is produced and the biomass is “used up”. In the unloading region, the “spent” biomass is removed via a transfer lock. Continuous operation is thus possible even in the case of biogas being generated in accordance with the principle of the methanation of solid matter.

Sealing problems frequently occur in particular in the case of large-scale fermenters for generating biogas from biomass, in particular at the corners and edges of the tanks and at the openings for loading and unloading purposes. Wet fermentation, in the case of which the liquefied biomass can be pumped into and out of the digestion tank, is thus known to use round tanks which have fewer corners and edges with sealing problems. The methanation of solid matter in large-scale fermenters does not make use of these round tanks on account of the problems during loading and unloading in batchwise operation. The transporting installation according to the present invention and the continuous operation which is thereby possible advantageously make it possible to use round tanks even for “dry fermentation”—claim 10.

The round construction of the large-scale fermenter reduces the sealing problems to a considerable extent since the outer wall and the inner wall are subjected merely to compressive and/or tensile loading. The rest of the sealing problems associated with corners and edges, however, are avoided. The design with an annular inner wall and an annular outer wall which encloses the inner wall results in an annular fermenter tank with an annular transporting channel. This annular cylinder is subdivided by a partition wall. The biomass is fed continuously in a loading region on one side of the partition wall and is discharged continuously in an unloading region on the other side of the partition wall, at the end of the transporting channel. The operations of feeding the fresh biomass in the loading region and of removing the spent biomass in the unloading region take place via transfer locks, for example by way of a liquid bath in the manner of a siphon.

The transporting installation used is preferably a transporting installation according to the present invention. The necessary fluid-control arrangement for activating the individual transporting cushions is coupled to the percolate-circulating arrangement and is preferably arranged in the interior within the annular inner wall—claim 12.

In particular if the biomass used stems from energy crops, the high liquid content of the biomass may result in excessive liquefaction of the biomass in the digestion tank. This would result in the transporting action of the transporting installation with transporting cushions being severely impaired. According to the advantageous configuration of the invention from claim 11, only partially fermented biomass is removed from between the loading and unloading regions, drained and guided back into the fermenter again. This measure, according to claim 13, considerably increases the concentration (number per unit of volume) of microorganisms generating methane gas, which has a positive effect on continued biogas generation—“biological turbocharger effect”.

The rest of the subclaims deal with advantageous configurations of the invention.

Further details, features and advantages of the invention are presented in the following description of exemplary embodiments, with reference to the drawings, in which:

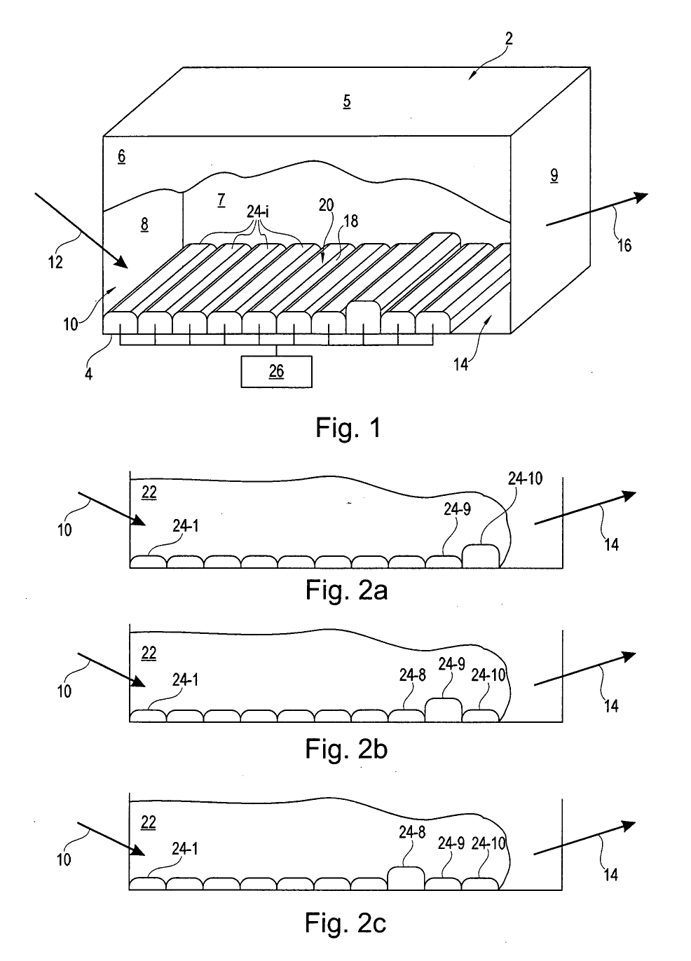

FIG. 1 shows a schematic illustration of an elongate large-scale fermenter, the base of which is covered by transporting cushions;

FIGS. 2a-2c show sectional illustrations depicting the wave movement generated by the transporting cushions;

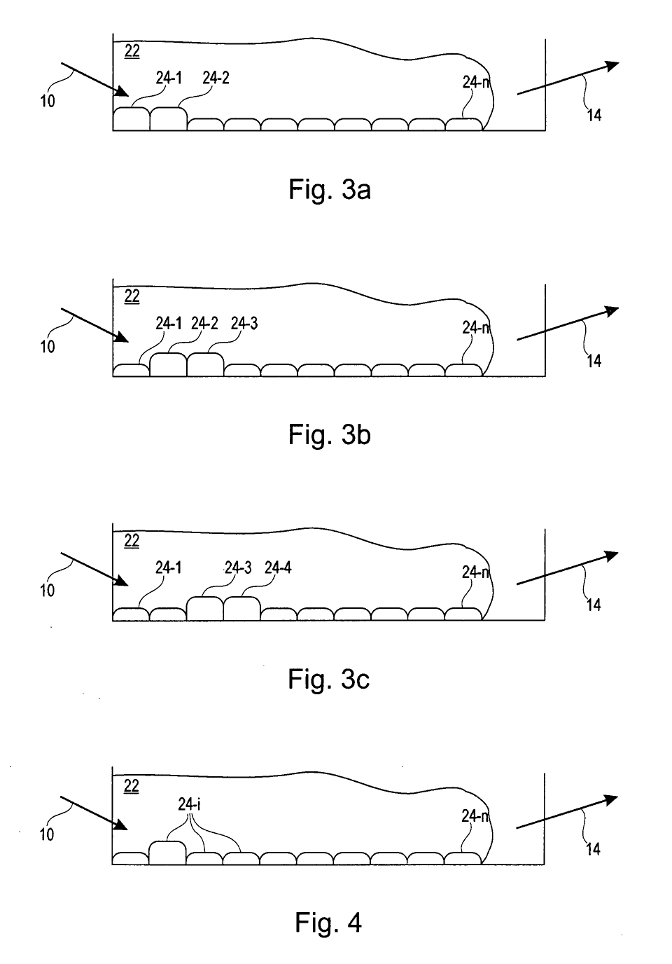

FIGS. 3a-3c show sectional illustrations portraying the wave movement generated by the transporting cushions using an alternative method of activation;

FIG. 4 shows an alternative configuration of the transporting cushions;

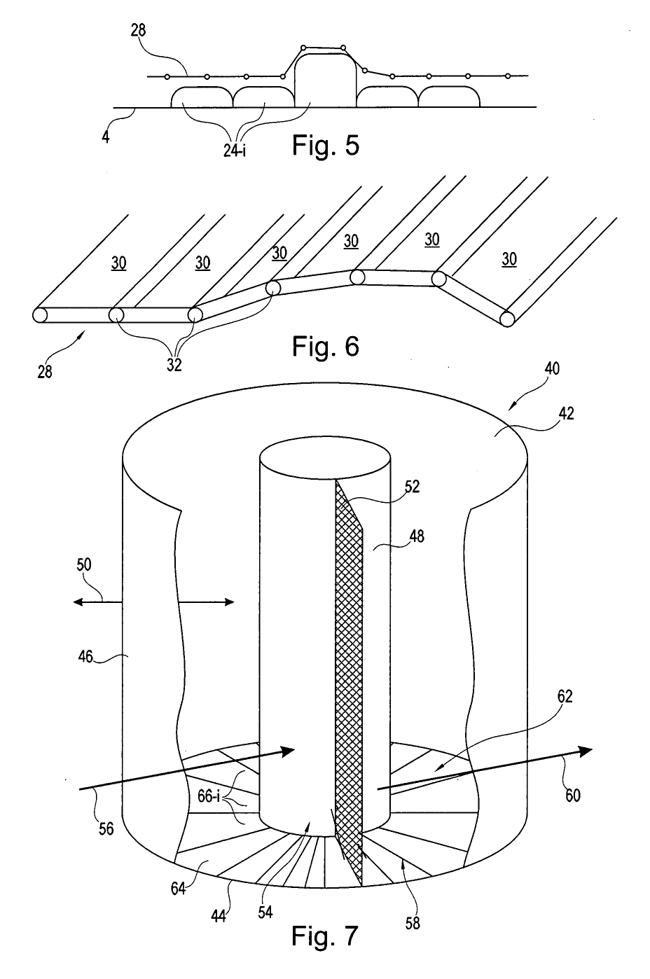

FIG. 5 shows an illustration corresponding to FIGS. 2a-2c and 3, the transporting cushions being provided with a covering which transmit the wave movement to the biomass located above;

FIG. 6 shows an illustration, in detail form, of the transporting-cushion covering;

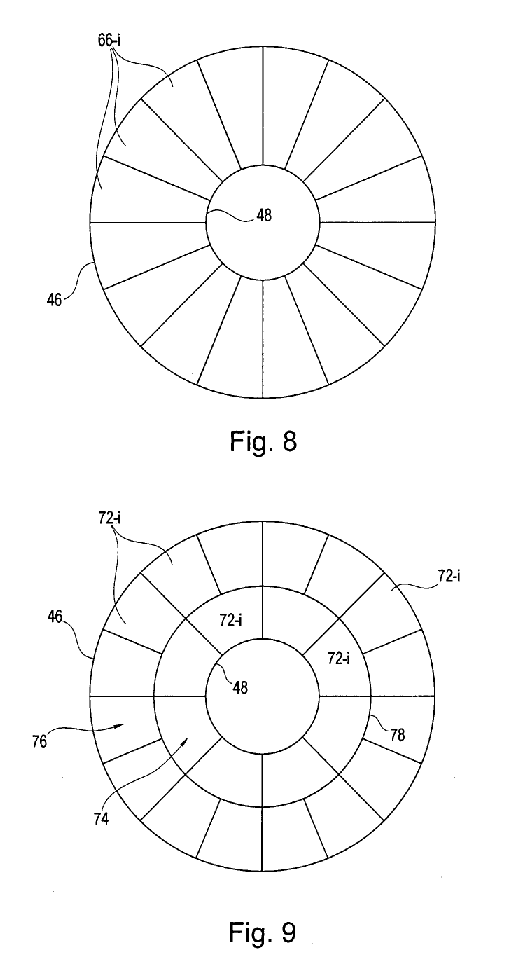

FIG. 7 shows a schematic illustration of a large-scale fermenter of round construction according to the present invention;

FIG. 8 shows a plan view of the base of the round large-scale fermenter with correspondingly shaped transporting cushions; and

FIG. 9 shows an alternative configuration and arrangement of the transporting cushions.

FIG. 1 shows, schematically, an elongate, cuboidal large-scale fermenter 2 with a rectangular base plate 4, a top wall 5, a right-hand side wall 6, a left-hand side wall 7, an end wall 8 and a rear wall 9. The large-scale fermenter 2 comprises, at one end, a loading region 10 with a loading arrangement 12—indicated by an arrow—which passes through the end wall 8 and, at the other end, an unloading region 14 with an unloading arrangement 16—likewise designated by an arrow—which passes through the rear wall 9. A transporting channel 18 which is bounded by the two side walls 6 and 7 is formed between the loading region 10 and unloading region 14.

The transporting channel 18 is provided with a transporting arrangement 20. Fresh biomass 22 is fed continuously in the loading region 10 by means of the loading arrangement 12. The transporting arrangement 20 conveys the biomass 22 to the unloading region 14 at the other end of the large-scale fermenter 2. Biomass 22 is removed from the unloading region 14 by means of the unloading arrangement 16.

The transporting arrangement 20 comprises a plurality of transporting cushions 24-i which are arranged on the base plate 4 directly adjacent to one another in the transporting channel 18. As can be seen from FIG. 1, the individual transporting cushions 24-i extend over the entire width of the large-scale fermenter 2 and are in the form of half-oval cylinders. The extent of the individual transporting cushions 14-i in the upward direction can be increased and reduced on a periodic basis by liquid being periodically fed and removed by means of a fluid-control arrangement 26. The feed of liquid to, and removal of liquid from, directly adjacent transporting cushions 24-i can generate a wave movement which conveys the biomass 22 from the loading region 10 to the unloading region 14.

The continuous transportation of biomass 22 by the transporting cushions 24-i is illustrated schematically in FIGS. 2a to 2c and 3a to 3c for a large-scale fermenter 2 filled with biomass 22. FIGS. 2 and 3, in order to depict the transporting wave movement, each show ten transporting cushions 24-1 to 24-10 distributed over the transporting channel 18. There is no transporting cushion provided in the unloading region 14. The illustration in FIGS. 3 and 4 is schematic and, in particular, is not true to scale.

In the first instance, according to FIG. 2a, liquid is pumped into the last transporting cushion 24-10 upstream of the unloading region 14 counter to the weight of the biomass 22 bearing on the last transporting cushion 24-10, and the biomass 22 bearing on the last transporting cushion 24-10 is raised and tips, in part, into the free unloading region 14. Thereafter, the liquid can escape again from the last transporting cushion 24-10 as a result of the weight of the biomass 22 bearing thereabove. According to FIG. 2b, liquid is then pumped into the last-but-one transporting cushion 24-9 and discharged again. The transporting cushion 24-8 is then pumped up and emptied again—FIG. 2c —until, finally, the first transporting cushion 24-1 is pumped up and emptied again (not illustrated). Thereafter, the operation begins again with the last transporting cushion 24-10. This generates a wave movement which conveys the biomass 22 continuously from the loading region 10 to the unloading region 14. With this method of activating the individual transporting cushions 24-i, the wave movement runs counter to the transporting direction. This method of activation is likely to be particularly suitable in the case of the biomass 22 having a particularly high dry-substance fraction.

In the case of biomass 22 having a lower dry-substance fraction and, possibly, a liquid level above the transporting cushions in which the dry substance of the biomass is floating, a wave movement in the transporting direction is likely to be more advantageous. This is illustrated schematically in FIGS. 3a to 3c. In the first instance, according to FIG. 3a, liquid is pumped into the first transporting cushion 24-1 in the loading region 10 counter to the weight of the biomass 22 bearing on the first transporting cushion 24-1 and the liquid above the first transporting cushion 24-1 is displaced. The second transporting cushion 24-2 is then pumped up with liquid—see FIG. 3a. Then—see FIG. 3b —the liquid is discharged from the first transporting cushion 24-1 and, at the same time, the third transporting cushion 24-3 is pumped up, while the second transporting cushion 24-2 remains pumped up. The next step—see FIG. 3c —is for the liquid to be discharged from the second transporting cushion and for the fourth transporting cushion 24-4 to be pumped up, while the third transporting cushion remains pumped up. This generates a “transporting wave” in the transporting direction which conveys the biomass 22 from the loading region 10 to the unloading region 14.

FIG. 4 shows, schematically, an alternative configuration of the transporting cushions 24-i such that the surface of the transporting cushions 24-i in the pumped-up state is inclined in the transporting direction. This configuration enhances the conveying action.

FIG. 5 shows a further configuration of the transporting arrangement according to the invention, this configuration differing from the abovedescribed embodiments in that the biomass 22, rather than resting directly on the transporting cushions 24-i, rests on a transporting-cushion covering 28, which rests on the transporting cushions 24-i. As can be seen from FIG. 5, the covering 28 comprises a plurality of board elements 30 which are connected to one another in an articulated manner via hinge or joint connections 32 in the manner of a roller blind for a window.

FIG. 7 shows a large-scale fermenter 40 of round construction with a circular-cylindrical digestion tank 42. The large-scale fermenter 40 comprises a planar base plate 44. The circular-cylindrical outer wall 46 extends upward from the base plate 44. The circular-cylindrical outer wall 46 encloses a circular-cylindrical inner wall 48 of smaller diameter. A covering (not illustrated) closes the space between the outer and inner walls 46, 48. The base plate 44, the outer wall 46, the inner wall 48 and the covering, which are connected to one another in a gas-tight manner, form the digestion tank 42.

The digestion tank 42 is subdivided in the interior by a partition wall 52. A loading region 54 with a loading arrangement 56 which passes through the outer wall 46 is provided on one side of the partition wall 52. An unloading region 58 with an unloading arrangement 60 which passes through the outer wall 46 is provided on the other side of the partition wall 52.

An annular transporting channel 62 which is bounded by the inner wall 48 and the outer wall 46 is formed between the loading region 54 and unloading region 58. The transporting channel 62 contains a transporting arrangement 64 of the type described with reference to FIGS. 2 to 5, this transporting arrangement comprising a plurality of transporting cushions 66-i which are arranged directly adjacent to one another on the base plate 54. As can be seen from FIG. 8, transporting cushions 66-i are approximately in the form of pieces of cake with cut-off tips, i.e. they are wider in the region of the outer wall 46 than in the region of the inner wall 48.

The double arrow 50 in FIG. 7 designates an unloading and loading arrangement which is arranged between the loading region 54 and the unloading region 58 such that it passes through the outer wall 46. Via the unloading and loading installation 50, the semi-fermented biomass 22 is removed from the digestion tank 42, drained and guided back into the digestion tank 42 again. The drainage can take place, for example, by means of a separator.

The transporting arrangement 64 having the transporting cushions 66-i is illustrated in a plan view in FIG. 8. The transporting wave movement is generated in a manner analogous to the embodiments according to FIGS. 1 to 6.

FIG. 9 shows an alternative embodiment of a transporting arrangement 70 in an illustration which corresponds to FIG. 7. The transporting arrangement 70 likewise comprises a plurality of transporting cushions 72-i which are distributed between an inner transporting-channel ring 74 and an outer transporting-channel ring 76. The inner and the outer transporting-channel rings 74, 76 are separated from one another by a central wall 78 arranged concentrically in relation to the inner and outer walls 48, 46.

The number of transport cushions 72-i in the outer transporting-channel ring 76 here is greater than in the inner transporting-channel ring 74. In the exemplary embodiment which is shown in FIG. 8, the number of transporting cushions 72-i in the outer transporting-channel ring 76 is double the number in the inner transporting-channel ring 74. This takes account of the fact that the transporting path in the outer transporting-channel ring 76 is longer than in the inner transporting-channel ring 74. The transporting wave movement is generated in a manner analogous to the embodiments according to FIGS. 1 to 6.

A transporting-cushion covering according to FIGS. 4 and 5 may also be provided for the transporting arrangements 64 and 70. It is also possible to provide for the configuration of the top side of the transporting cushions according to FIG. 5.

The large-scale fermenter for continuous operation according to the invention is particularly suitable for biomass from energy crops since, on account of its homogeneity, this biomass is easy to convey by the transporting arrangement according to the invention.

### LIST OF DESIGNATIONS

2 Large-scale fermenter
4 Base plate
5 Top wall
6 Left-hand side wall
7 Right-hand side wall
8 End wall
9 Rear wall
10 Loading region
12 Loading arrangement
14 Unloading region
16 Unloading arrangement
18 Transporting channel
20 Transporting arrangement
22 Biomass
24-i Transporting cushion
26 Fluid-control arrangement
28 Transporting-cushion covering
30 Board elements
32 Hinge or joint connection
40 Large-scale fermenter of round construction
42 Digestion tank
44 Base plate
46 Outer wall
48 Inner wall
50 Unloading and loading arrangement
52 Partition wall
54 Loading region
56 Loading arrangement
58 Unloading region
60 Unloading arrangement
62 Transporting channel
64 Transporting arrangement
66-i Transporting cushion
70 Transporting arrangement
72-i Transporting cushion
74 Inner transporting-channel ring
76 Outer transporting-channel ring
78 Central wall

1. A transporting arrangement for transporting biomass from energy crops in a fermenter for generating biogas, comprising:
a transporting channel with mutually opposite side walls connected to one another via a base surface,
a plurality of transporting cushions distributed over the transporting channel, the plurality of transporting cushions configured for being filled with fluid and arranged beneath the biomass to be transported, and
a fluid-control arrangement for successively filling and emptying successive transporting cushions to generate a wave movement which moves the biomass through the transporting channel,
wherein the fluid for filling the transporting cushions is a liquid.

2. The transporting arrangement as recited in claim 1, wherein the liquid for filling the transporting cushions is a percolate.

3. The transporting arrangement as recited in claim 1, wherein the transporting cushions are arranged in abutment against one another.

4. The transporting arrangement as recited in claim 1, wherein provided over the transporting cushions is a transporting-cushion cover by means of which wave movement which can be generated by the transporting cushions is transmitted to the biomass.

5. The transporting arrangement as recited in claim 1, wherein the transporting cushions are fastened on the base surface.

6. The transporting arrangement as recited in claim 3 or 4, wherein the transporting cushions are assigned to one another in pairs and are fastened opposite one another on the side walls.

7. The transporting arrangement as recited in claim 1, wherein the transporting cushions can generate a wave movement counter to the transporting direction.

8. The transporting arrangement as recited in claim 1, wherein the transporting cushions can generate a wave movement in the transporting direction.

9. A large-scale fermenter for generating biogas from biomass in accordance with the principle of the methanation of solid matter, comprising:
a gas-tight digestion tank with a base plate,
a loading region with a loading arrangement which passes through the gas-tight digestion tank and is intended for feeding fresh biomass continuously into the digestion tank,
an unloading region with an unloading arrangement which passes through the gas-tight digestion tank and is intended for removing spent biomass from the digestion tank,
a transporting arrangement for transporting the biomass from the loading region to the unloading region, and
a biogas-removal connection.

10. The large-scale fermenter as recited in claim 9, the digestion tank comprising:
a gas-tight circular-cylindrical outer wall which extends upwards from the base plate,
a gas-tight circular-cylindrical inner wall which extends upward from the base plate,
a gas-tight covering is connected to the outer and inner walls, the base plate, outer wall, inner wall and covering defining a cylindrical digestion tank therebetween for accommodating the biomass, and
a partition wall which extends upward from the base plate,
wherein the loading arrangement passes through the outer wall on one side of the partition wall, and
wherein the unloading arrangement passes through the outer wall on the other side of the partition wall.

11. The large-scale fermenter as recited in claim 9, wherein a central wall extends upward from the base plate between the inner wall and outer wall, forming an inner transporting-channel ring between the inner wall and central wall and forming an outer transporting-channel ring between the outer wall and central wall.

12. The large-scale fermenter as recited in one of claims 9 to 11, having a percolate-circulating arrangement which has a percolate-storage tank arranged within the cylindrical wall.

13. The large-scale fermenter as recited in one of claims 9 to 11, having at least one removal and loading arrangement which is arranged between the loading region and the unloading region in order for biomass to be removed from the digestion tank, drained and fed back to the digestion tank.
